(19) United States
(12) Patent Application Publication
(10) Pub. No.: US 2010/0252479 A1
(43) Pub. Date: Oct. 7, 2010

(54) MEDICATION DISPENSING SYSTEMS AND METHODS

(76) Inventor: Kenneth M. Corroon, Greenwich, CT (US)

Correspondence Address: PILLSBURY WINTHROP SHAW PITTMAN LLP, ATTENTION: DOCKETING DEPARTMENT, P.O BOX 10500, McLean, VA, 22102, US

(21) Appl. No.: 12/416,889

(22) Filed: Apr. 1, 2009

(51) Int. Cl. B65D 83/04; A61B 19/02

(52) U.S. Cl. 206/534; 206/528

(57) ABSTRACT

Systems and methods for packaging, identifying and dispensing medicines are described. The medicines may include homeopathic and other products. A package may include a container is having a port through which medicine is dispensed. A sleeve partially encloses the container and is slidable along a portion of a surface of the container between two positions. The port is accessible when the sleeve is in one of the two positions and inaccessible when the sleeve is in the other position. The sleeve may include a cover that prevents unassisted extraction of the objects from the container where the cover may be a semi-adhesive tape or similar. An identifier and color coding may be provided that distinguishes the contents from the contents of other packages.

### BACKGROUND OF THE INVENTION

1. Field of the Invention

The present invention relates generally to packaging and more particularly to the packaging of medications.

2. Description of Related Art

Homeopathy has been used for well over 200 years, developing a remarkable record of safety and generating a significant body of knowledge. Today, homeopathy is the second most widely used system of medicine in the world. It is used by millions of patients in more than 70 countries, recommended by more than 500,000 healthcare providers and regulated by the Food and Drug Administration (“FDA”) in the United States. In order for the FDA to accept a homeopathic medicine, the product ingredients must be identified in the Homeopathic Pharmacopoeia of the United States (“HPUS”) or generally accepted as homeopathic under the principles of homeopathy.

Although the basic principle of homeopathy, ‘like cures like’, was recognized by the ancient Greek physician of the Age of Pericles, Hippocrates of Kos (ca. 460 BC-ca. 370 BC), it was the German physician Dr. Samuel Hahnemann who in the late 1700s is credited with its development as a viable medical practice. Disenchanted with what he saw as barbaric medical practices in the eighteenth century, Hahnemann came across ‘A Treatise on Materia’ by Dr. Edward Cullen in which Cullen stated that quinine was an effective treatment for malaria. Intrigued, Hahnemann investigated the effects of quinine on himself and his friends and found that, in a healthy person, quinine produced the same symptoms as the disease itself—fever, sweating and shivering—whereas in an ill person it acted as a cure.

Each homeopathic medicine is directed to treatment of specific conditions.

### BRIEF SUMMARY OF THE INVENTION

Certain embodiments of the present invention comprise systems and methods for packaging, identifying and dispensing medicines, including homeopathic products. In one embodiment a package according to certain aspects of the invention may be configured for quick, accurate identification of contents, intended use and for rapid distribution of doses.

In certain embodiments, a container is provided that is configured to house a plurality of objects such as pills, caplets, gel caps and capsules. The container typically has at least one port for dispensing in a measured quantity of the objects and a sleeve that partially encloses the container. The sleeve can be slidable along a portion of a surface of the container between two positions, and the port is accessible when the sleeve is in one of the two positions and inaccessible when the sleeve is in the other position.

In certain embodiments, a port comprises an opening shaped to control rate of dispensing of the objects and may include a cover that prevents unassisted extraction of the objects from the container. That is, the medicine is typically confined within the container until a user exerts a pressure, a force, pulls or otherwise manipulates a pill or other dosage of medicine from the container. A cover can comprise a semi-adhesive tape and, in one example, the opening is substantially diamond shaped.

In certain embodiments, the container carries an identifier visible when the sleeve is in the second position. In one example, the identifier is visible when it is aligned with a hole in the sleeve. Furthermore, the container may resist movement from the second position when the identifier is visible when, for example, the identifier is provided on a raised surface on the container that protrudes through the hole when the identifier is fully visible. In that regard, a portion of the raised surface may be rotatable about an axis perpendicular to the plane of the hole and, typically passing through a point within the hole, when the sleeve is in the second position. The rotatable portion of the raised surface may be rotated to lock the package with the sleeve in the second position.

In certain embodiments, the objects maintained within the container are medications. Other ports may be provided to permit dispensing different types of medication from each port. The medication types may be provided in lozenge, pill, gel cap, caplet, capsule and other forms. In certain embodiments, one or more of the container and the sleeve are color coded based on characteristics of the one or more medications.

In certain embodiments methods for maintaining medications are provided in packages constructed according to certain aspects of the invention. These methods may include providing a plurality of color coded packages, color coding the container or sleeve to identify the intended contents of the container, charging the containers with medications corresponding to the color code and labeling each package with a description of its contents. Some embodiments comprise the step of providing each container with an identifier that enables a user to determine the type of the contents of the each package. The container may carry an identifier visible when the sleeve is in the second position.

Certain embodiments of the invention provide systems and methods for packaging and dispensing medications, particularly in lozenge, pill, gel cap, caplet, capsule and other forms. Some of these embodiments comprise a container for containing a medicine in solid form. Some of these embodiments comprise a sleeve that partially encloses the container and that is slidable along a portion of a surface of the container between a first position and a second position. Some of these embodiments comprise a port configured to dispense the medicine from the container. In some of these embodiments, the port is accessible when the sleeve is in the first position and inaccessible when the sleeve is in the second position. In some of these embodiments, the port has a shape and size selected to control dispensing of the medicine, and further comprising a cover for the port that prevents unassisted extraction of the medicine from the container.

### BRIEF DESCRIPTION OF THE DRAWINGS

FIG. 1A depicts a closed package according to certain aspects of the invention.

FIG. 1B depicts an opened package according to certain aspects of the invention.

FIG. 2A illustrates a first container portion of a package according to certain aspects of the invention.

FIG. 2B illustrates a second container portion of a package according to certain aspects of the invention.

FIG. 3 depicts an alternative package according to certain aspects of the invention.

FIG. 4A depicts a button on a container according to certain aspects of the invention.

FIG. 4B depicts a locking mechanism on a button.

FIG. 4C illustrates the placement of an identifier according to certain aspects of the invention.

FIG. 5 depicts a form of container before final assembly according to certain aspects of the invention.

FIGS. 6A-6C show different versions of a sleeve according to certain aspects of the invention.

### DETAILED DESCRIPTION OF THE INVENTION

Embodiments of the present invention will now be described in detail with reference to the drawings, which are provided as illustrative examples so as to enable those skilled in the art to practice the invention. Notably, the figures and examples below are not meant to limit the scope of the present invention to a single embodiment, but other embodiments are possible by way of interchange of some or all of the described or illustrated elements. Wherever convenient, the same reference numbers will be used throughout the drawings to refer to same or like parts. Where certain elements of these embodiments can be partially or fully implemented using known components, only those portions of such known components that are necessary for an understanding of the present invention will be described, and detailed descriptions of other portions of such known components will be omitted so as not to obscure the invention. In the present specification, an embodiment showing a singular component should not be considered limiting; rather, the invention is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the components referred to herein by way of illustration.

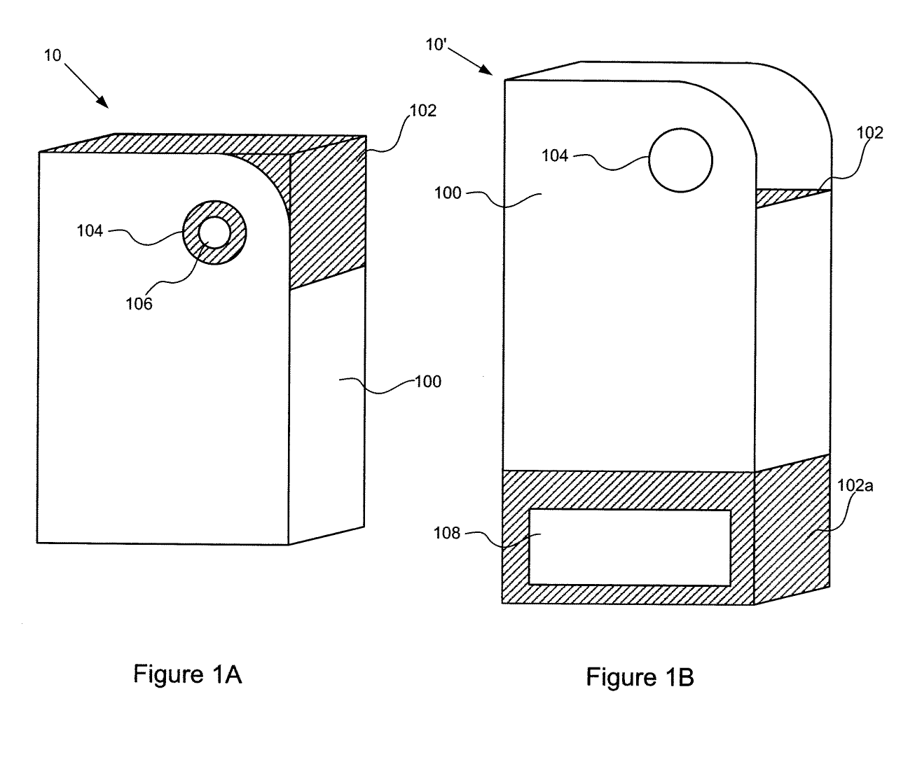

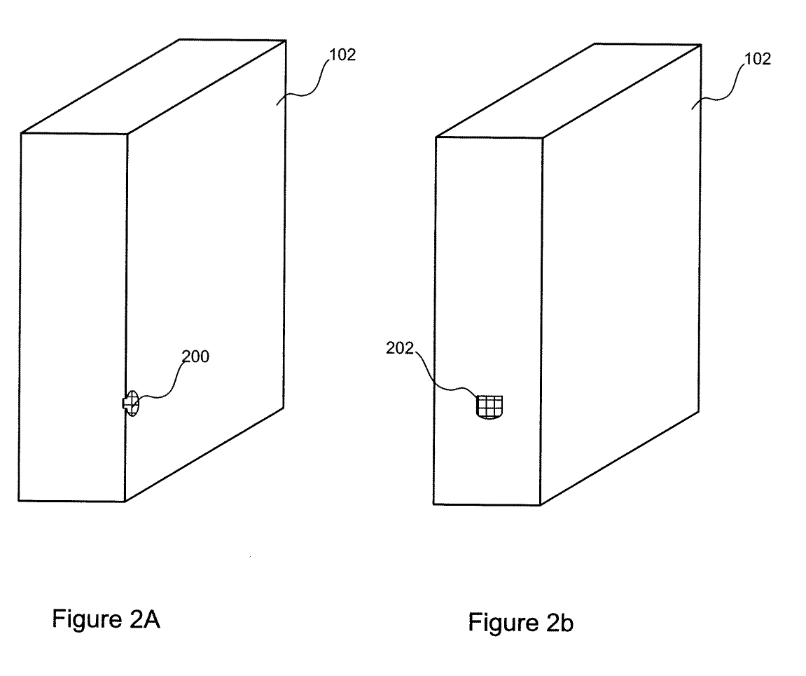

Certain embodiments of the present invention comprise systems and methods for packaging, identifying and dispensing products that may include medicines for treating certain conditions. FIGS. 1A and 1B depicts an example of packaging constructed according to certain aspects of the present invention. FIG. 1A shows a package in a first state 10 and FIG. 1B shows the package in a second state 10′. In the first state, access to medicine or other contents is prevented. Contents of the package 10 are typically in solid form, such as pill, caplet, capsule (e.g. gelatin capsule), lozenge, gel cap, and other forms. Contents of the package 10 are maintained in a box or container 102 which is partially enclosed by a sleeve 100. In the second state 100′, container 102 is pushed downwards relative to the sleeve 100 as shown in FIG. 1B, thereby exposing a lower portion 102a of container 102. The lower portion 102a may also be exposed by sliding sleeve 100 away from the container 102. As will be described later, exposing lower portion 102a unlocks access or otherwise enables access to the contents container 102. For example, an opening (see FIGS. 2A and 2B) may become accessible when lower portion 102a is exposed.

In certain embodiments, the package in both states (10 and 10′) may display identifications of the contents of container and other information related to the contents. The identifications and other information may be visible or partially visible in both states 10 and 10′. In some embodiments, portions of the information and/or identification may be visible only in one of the states 10 or 10′. For example, a portion of the package may color coded as depicted in the drawings, where color coding may be associated with the color of the shaded portions of container 102. The color coding may indicate the type and/or form of the contents of container 102. A color coding key may be provided on a portion 108 that is only visible in state 10′ to avoid confusion that could be caused by listing a plurality of potential container content types. In one example, jetlag relief medication may be provided in a teal colored container 102. In the latter example, an icon 106 of a jet airplane may also be provided on the container that is visible through a hole 104 in sleeve 100 when package is in state 10. When the package is in state 10′, the icon 106 may be obscured or hidden, while a legend 108 becomes viewable that shows a teal colored airplane similar to icon 106 in a field of the legend; other fields of the legend may depict other color coded icons that are associated with other container contents. Thus an orange glass icon may be depicted as a hangover medication, indicating that an orange colored container 102 having a glass for icon 106 would be easily distinguished from the described jetlag medication.

Accordingly, a range of medications can be provided in packages having identical form and function but differentiated by color coding and icons or images. Certain areas of the package may be reserved for information and identification that is required by regulation and statutes of the jurisdictions in which the product is to be marketed. Thus, in certain embodiments, portions of the sleeve surface area can be reserved for government mandated warnings, ingredient information and directions for use. Other parts of the sleeve may be reserved for product identification, description of the contents and distributor/manufacturer trademarks, etc. The sleeve is typically chosen for mandated and marketing information because it is visible in both states 10 and 10′.

In certain embodiments, information may be provided in combinations of graphics and text elements. Graphics elements can include icons 106, barcodes, logos, instructional graphics such as arrows, scissors and so on, while text may be provided in one or more languages and forms. For example, packaging may include written descriptions of content and directions as well as inscribed or embossed Braille descriptions.

FIGS. 2A and 2B provide an isolated view of inner container 102 of a package constructed according to certain aspects of the invention. This inner container typically encloses the contents of the package. In one example, the contents can be homeopathic tablets that are accessible through one or more ports 200, 202 provided in the container 102. The location and size of port 200 or 202 is selected based on ergonomics and selection typically takes into account the size, weight and shape of the medication to be dispensed, expected dexterity of users of the medication and other factors. In one example, medication for the treatment of arthritis or rheumatism may be provided in larger pill forms for easier handling and this sizing may affect the form, location and size of port 200 or 202. In another example, medication for a condition may include two different pills that may be dispensed through different ports from an internally divided container 102. In this latter example, two instances of port 200 may be provided on opposite surfaces of container 102 to enable easy access to both pills. Of course, other methods for distribution of multiple pills may be employed and it is contemplated that a single port 200 or 202 can be equipped with a mechanism that selectively dispenses desired combinations of different medications upon each opening of the port 200 or 202.

In certain embodiments, the shape of the port 200 is selected to control dispensing and/or a rate of dispensing of the contents. The shape and size of an opening in the port 200 may, for example, limit the number of pills that can protrude through the opening to enable extraction. For example, a suitably shaped and sized opening may allow a portion of the pill to easily pass through the opening without allowing unassisted exit from container 102: the pill may need to be pulled to enable its escape from the container. The ease and extent to which the pill can exit may be determined by a combination of additional factors such as form factor of the pill and presence of a barrier material covering the opening. In one example, port 200 comprises a customized diamond-shaped opening and a flap or other cover that maintains the contents within container 102 until application of pressure, opening of the flap or other action taken by a user of the package. For example, a semi-adhesive tape may be used to seal the port 200 or 202 between usages. In another example, a polymer sheet material may be provided such that an opening in the sheet yields under applied pressure to allow pills to exit the container. In another example, a slide may be hinged around an edge of port 202 such that the slide, when extended, can conduct pills through port 202 from the interior to the exterior of the container 102. In certain embodiments, a port comprises an opening shaped to control rate of dispensing of the objects and may include a cover that prevents unassisted extraction of the objects from the container. That is, the medicine can be confined within the container until a user exerts a pressure, force sufficient to overcome the resistance to pulling or pushing a pill from the container 102.

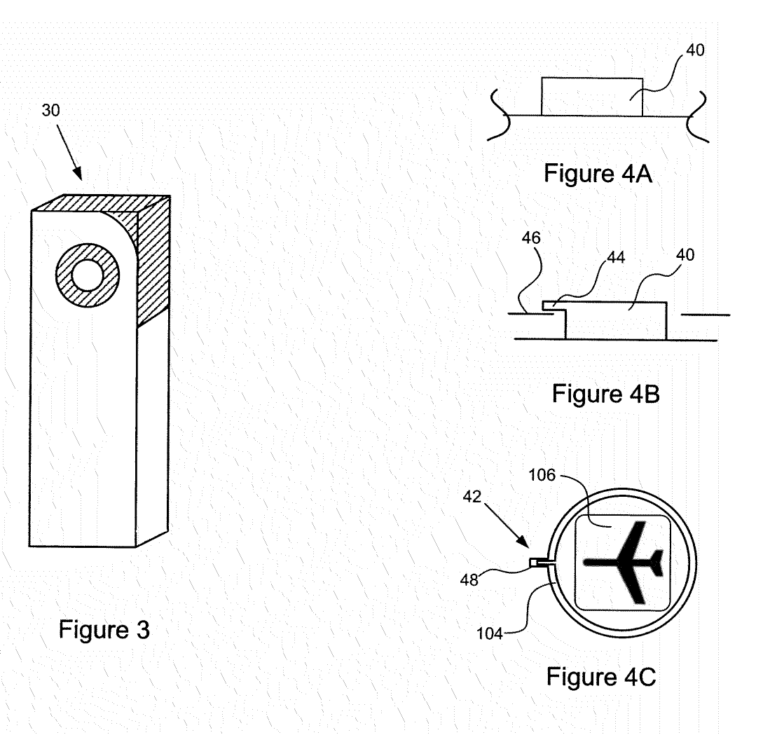

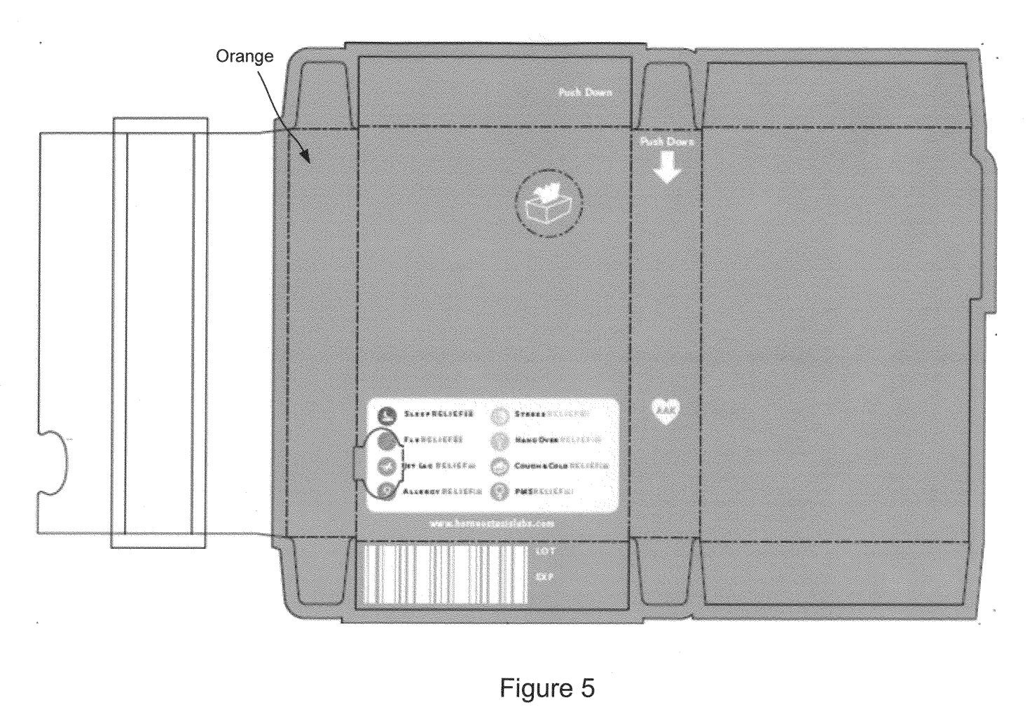

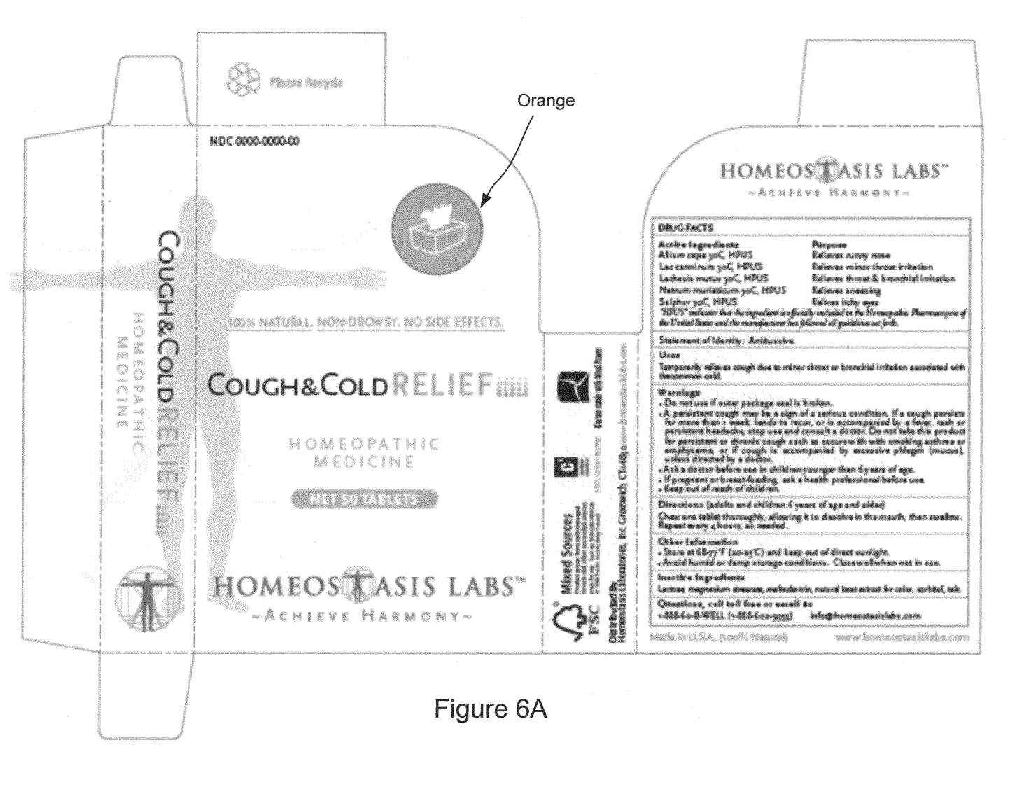

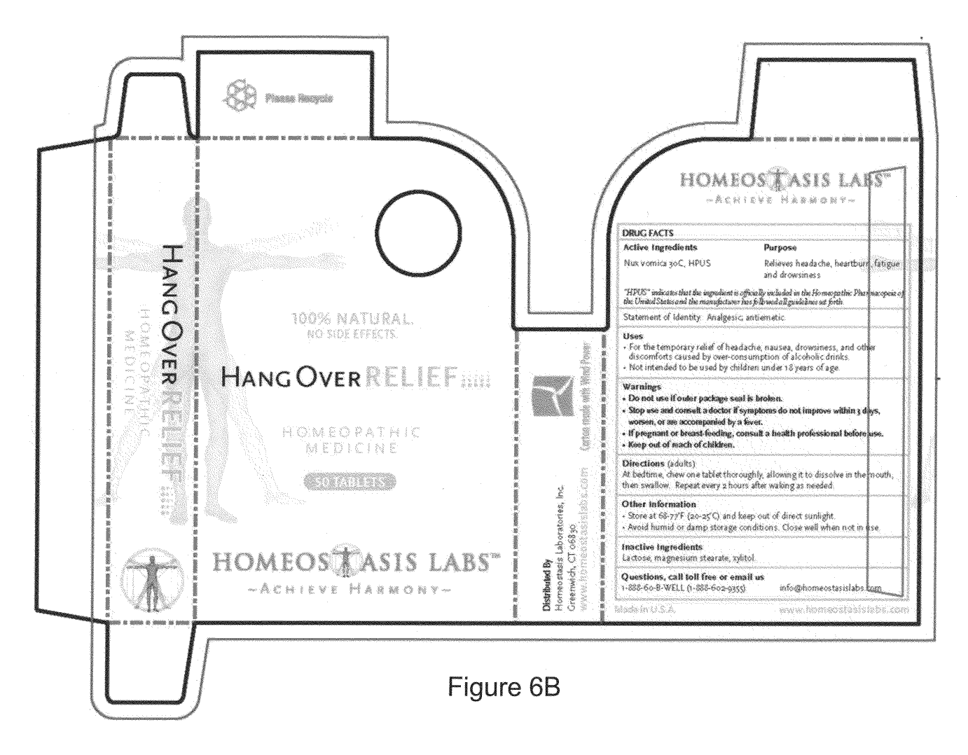

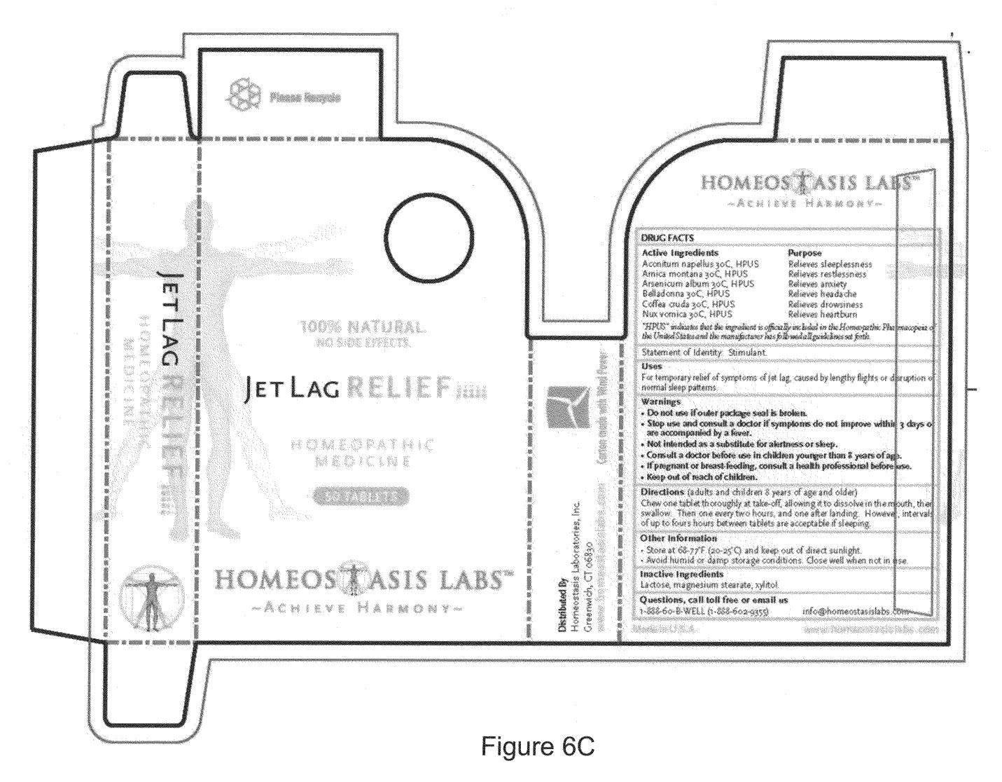

In certain embodiments, the package can easily be manufactured to provide cost-effective, secure packaging of medications. FIGS. 3 and 4 depict components used to construct the two part package 10 and 10′ described above. FIG. 3 shows the form of a sheet material that can be used to create sleeve 100. Sleeve 100 can be produced from any material suited to the application and environment in which the contents will be dispensed and consumed. For example, sleeve 100 and container 102 may both be fabricated from card or paper stock for most applications. In one example, major components of a container may be cost-effectively constructed entirely from card stock of sufficient weight to provide the mechanical properties necessary to protect the contents during use and transportation. FIG. 4 shows a form that may be cut from a sheet of card or other material in order to form above-described inner container 102.

In certain embodiments, paper, card or other base material may be coated in order to obtain enhanced protection of the contents from fluid and humidity and/or to strengthen the physical structure of the packaging. Coatings can include latex and polymers. In one example, a paper or card form may be cut and printed with graphics and information directed to the intended content of the container 102. One or more surfaces can be coated with a fluid, suspension, paste or other suitable material that comprises monomers which upon the application of ultra-violet light, polymerize to form a hardened coating having desired properties. The properties may include one or more of strength, impermeability, transparency, smoothness, color, tint, shade and so on. A coating may be applied before or after final assembly. In some embodiments, only the outer surfaces of the packaging are coated. However, some benefit may be accrued from coating the inside of container 102 and/or the inside of sleeve 100. In certain embodiments, portions of the packaging may be uncoated in order to achieve a desired finish that may exhibit a matte or otherwise textured surface, to improve hypoallergenic characteristics, etc.

The volume of the package and the dimensions of its component parts may be selected based on application. In one example, the closed container shown in FIGS. 1A and 1B may be constructed with a 3.5″ height, a 2.5″ width and a 0.5″ depth (approximately). This latter configuration is typically suitable for general use, being pocket-sized and/or purse-sized. In another example, illustrated in FIG. 3, a package intended for limited-dosage use may be substantially smaller in one or more dimensions. For example, width may be reduced to 0.5″ and height can be reduced based on the number of doses to be carried and the number of pills, tablets, caplets, etc. that are required for a single dose.

Other aspects of the package 10 may be modified to accommodate particular usage. With reference to FIG. 4A for example, icon 106 (as identified in FIG. 1A) may be provided on a raised platform 40 that is dimensioned to engage with hole 104, thereby creating a locking mechanism or point of resistance to opening that prevents inadvertent extension of inner container 102. The raised platform 40 may be characterized as a button and it will be appreciated that such raised platform 40 may be provided from a material different to that used for manufacturing inner container 102. In some embodiments, raised platform 40 may be combined with other mechanisms to “child-proof” the package. For example, part of icon 106 or raised platform 40 may be rotatable such that orientation of icon 106 in a particular configuration is required to release inner container 102. In the example of FIG. 4B, a key and slot lock 42 is used such that key 44 of rotatable raised platform 40 is obstructed by the body 46 of the sleeve, unless aligned with slot 48. Other mechanisms may be used to provide locking functionality. For example, a degree of force may be required to deform raised platform 40 sufficiently for raised platform to fit through hole 104. Force may be required at two or more points to obtain deformation.

In certain embodiments, the package is charged with contents and sealed in a transparent wrapper for shipping. The transparent wrapper is typically a cellophane, polythene or polycarbonate sheet material that is sealed to protect the package and its content from environmental damage and/or tampering. The method of sealing can include the application of glue, heat, pressure or a combination of these and other techniques.

### Additional Descriptions of Certain Aspects of the Invention

The foregoing descriptions of the invention are intended to be illustrative and not limiting. For example, those skilled in the art will appreciate that the invention can be practiced with various combinations of the functionalities and capabilities described above, and can include fewer or additional components than described above. Certain additional aspects and features of the invention are further set forth below, and can be obtained using the functionalities and components described in more detail above, as will be appreciated by those skilled in the art after being taught by the present disclosure.

Certain embodiments of the invention provide systems and methods for a package. Some of these embodiments comprise a container configured to house a plurality of objects. In some of these embodiments, the container has at least one port for dispensing in a measured quantity of the objects. Some of these embodiments comprise a sleeve that partially encloses the container. In some of these embodiments, the sleeve is slidable along a portion of a surface of the container between two positions. In some of these embodiments, the port is accessible when the sleeve is in one of the two positions and inaccessible when the sleeve is in the other position. In some of these embodiments, the sleeve is slidable along a portion of a surface of the container between a first position and a second position, wherein the port is accessible when the sleeve is in the first position and inaccessible when the sleeve is in the second position.

In some of these embodiments, the at least one port comprises in an opening shaped to control rate of dispensing of the objects. In some of these embodiments, the at least one port includes a cover that prevents unassisted exit of the objects from the container. In some of these embodiments, the cover comprises a semi-adhesive tape. In some of these embodiments, the opening is substantially diamond shaped. In some of these embodiments, the container carries an identifier visible when the sleeve is in the second position. In some of these embodiments, the identifier is visible when aligned with a hole in the sleeve. In some of these embodiments, the container resists movement from the second position when the identifier is visible. In some of these embodiments, the identifier is provided on a raised surface on the container that protrudes through the hole when the identifier is fully visible. In some of these embodiments, a portion of the raised surface is rotatable about an axis perpendicular to a point in the hole when the sleeve is in the second position. In some of these embodiments, the rotatable portion of the raised surface may be rotated to lock the package with the sleeve in the second position.

In some of these embodiments, the objects include one or more medications. In some of these embodiments, the at least one port includes two different ports, each of the two ports dispensing a type of medication different from the other port, wherein medication types include pills, caplets and gelatin capsules. In some of these embodiments, one or more of the container and the sleeve are color coded based on characteristics of the one or more medications.

Certain embodiments of the invention provide systems and methods for maintaining medications. Some of these embodiments comprise the step of providing a plurality of color coded packages. In some of these embodiments, each package includes a container configured to house a plurality of objects. In some of these embodiments, the container includes at least one port for dispensing in a measured quantity of the objects. In some of these embodiments, each package includes a sleeve that partially encloses the container and that is slidable along a portion of a surface of the container between two positions. In some of these embodiments, the port is accessible when the sleeve is in a first of the two positions and inaccessible when the sleeve is in the second position. In some of these embodiments, a color coding identifies the intended contents of the container. Some of these embodiments comprise charging the containers with medications corresponding to the color code. Some of these embodiments comprise labeling each package with a description of its contents. Some of these embodiments comprise the step of providing each container with an identifier that enables a user to determine the type of the contents of the each package. In some of these embodiments, the container carries an identifier visible when the sleeve is in the second position. In some of these embodiments, the at least one port comprises in an opening shaped to control rate of dispensing of the objects. In some of these embodiments, the at least one port includes two different ports. In some of these embodiments, each of the two ports dispensing a type of medication different from the other port. In some of these embodiments, the identifier is an icon provided on a raised surface on the container that is configured to protrude through the hole when the sleeve is in the second position thereby resisting transition of the sleeve from the second position to the first position.

Certain embodiments of the invention provide systems and methods for packaging and dispensing medications, particularly in pill, caplet and/or capsule form. Some of these embodiments comprise a container for containing a medicine in solid form. Some of these embodiments comprise a sleeve that partially encloses the container and that is slidable along a portion of a surface of the container between a first position and a second position. Some of these embodiments comprise a port configured to dispense the medicine from the container. In some of these embodiments, the port is accessible when the sleeve is in the first position and inaccessible when the sleeve is in the second position. In some of these embodiments, the port has a shape and size selected to control dispensing of the medicine, and further comprising a cover for the port that prevents unassisted extraction of the medicine from the container.

Although the present invention has been described with reference to specific exemplary embodiments, it will be evident to one of ordinary skill in the art that various modifications and changes may be made to these embodiments without departing from the broader spirit and scope of the invention. Accordingly, the specification and drawings are to be regarded in an illustrative rather than a restrictive sense.

What is claimed is:

1. A package, comprising:
a container configured to house a plurality of objects, the container including at least one port for dispensing in a measured quantity of the objects; and
a sleeve that partially encloses the container and that is slidable along a portion of a surface of the container between a first position and a second position, wherein the port is accessible when the sleeve is in the first position and inaccessible when the sleeve is in the second position.

2. The package of claim 1, wherein the at least one port comprises in an opening shaped to control rate of dispensing of the objects.

3. The package of claim 1, wherein the at least one port includes a cover that prevents unassisted exit of the objects from the container.

4. The package of claim 3, wherein the cover comprises a semi-adhesive tape.

5. The package of claim 3, wherein the opening is substantially diamond shaped.

6. The package of claim 1, wherein the container carries an identifier visible when the sleeve is in the second position.

7. The package of claim 6, wherein the identifier is visible when aligned with a hole in the sleeve.

8. The package of claim 7, wherein the container resists movement from the second position when the identifier is visible.

9. The package of claim 7, wherein the identifier is provided on a raised surface on the container that protrudes through the hole when the identifier is fully visible.

10. The package of claim 9, wherein a portion of the raised surface is rotatable about an axis perpendicular to a point in the hole when the sleeve is in the second position, and wherein the rotatable portion of the raised surface may be rotated to lock the package with the sleeve in the second position.

11. The package of claim 1, wherein the objects include one or more medications.

12. The package of claim 11, wherein the at least one port includes two different ports, each of the two ports dispensing a medication different from the other port, wherein the one or more medications includes one or more of pills, caplets, capsules, lozenge and gel caps.

13. The package of claim 11, wherein one or more of the container and the sleeve are color coded based on characteristics of the one or more medications.

14. A method for maintaining medications, comprising:
providing a plurality of color coded packages, each package including
a container configured to house a plurality of objects, the container including at least one port for dispensing in a measured quantity of the objects,
a sleeve that partially encloses the container and that is slidable along a portion of a surface of the container between two positions, wherein the port is accessible when the sleeve is in a first of the two positions and inaccessible when the sleeve is in the second position, and
a color coding identifying the contents of the container;
charging the containers with medications corresponding to the color code; and
labeling each package with a description of its contents.

15. The method of claim 14, and further comprising providing each container with an identifier that enables a user to determine the type of the contents of the each package, wherein the container carries an identifier visible when the sleeve is in the second position.

16. The package of claim 14, wherein the at least one port comprises in an opening shaped to control rate of dispensing of the objects, the objects including one or more of a pill, a caplet, a capsule, a lozenge and a gel cap.

17. The package of claim 16, wherein the at least one port includes two different ports, each of the two ports dispensing a type of medication different from the other port.

18. The package of claim 15, wherein the identifier is an icon provided on a raised surface on the container that is configured to protrude through the hole when the sleeve is in the second position thereby resisting transition of the sleeve from the second position to the first position.

19. A medication package and dispenser, comprising:
a container for containing a medicine in solid form;
a sleeve that partially encloses the container and that is slidable along a portion of a surface of the container between a first position and a second position; and
a port configured to dispense the medicine from the container, wherein the port is accessible when the sleeve is in the first position and inaccessible when the sleeve is in the second position.

20. The medication package and dispenser of claim 19, wherein the port has a shape and size selected to control dispensing of the medicine, and further comprising a cover for the port that prevents unassisted extraction of the medicine from the container.
